(19) United States
(12) Patent Application Publication
(10) Pub. No.: US 2007/0049872 A1
(43) Pub. Date: Mar. 1, 2007

(54) Syringe clip

(76) Inventors: Philip Watts, Long Beach, CA (US); Brian F. Pockett, San Marcos, CA (US); David R. Koos, San Diego, CA (US); John A. Griecci, Redondo Beach, CA (US)

Correspondence Address: THE LAW OFFICE OF JOHN A. GRIECCI, 703 PIER AVE., SUITE B #657, HERMOSA BEACH, CA, 90254, US

(21) Appl. No.: 11/480,762

(22) Filed: Jul. 3, 2006

Related U.S. Application Data
(60) Provisional application No. 60/696,310, filed on Jul. 1, 2005.
(60) Provisional application No. 60/720,135, filed on Sep. 23, 2005.

(51) Int. Cl. A61M 5/00

(52) U.S. Cl. 604/187

(57) ABSTRACT

A clip having a compression surface and a clamping mechanism to compress an outer surface of a syringe barrel around a sealing portion. The clamping mechanism can comprise a rigid c-shape body, a flexible body that latches to clamp, or a flexible strap. The clip can alternatively clamp on to two flanges of a plunger, and have a protrusion that locks onto a syringe barrel flange.

This application claims the benefit of U.S. provisional Application No. 60/696,310, filed Jul. 1, 2005, and of U.S. provisional Application No. 60/720,135, filed Sep. 23, 2005. Each of the aforementioned application(s) are incorporated herein by reference for all purposes.

This invention relates generally to syringes, and more particularly, to a syringe clip configured to lock a syringe plunger's location with respect to a syringe barrel.

### BACKGROUND

Typically, a syringe is comprised of a barrel and a plunger. The barrel includes a hollow, barrel cylinder, a connector portion on a distal end of the barrel, and a flange on a proximal end of the barrel. The barrel is in the form of a cylinder defining a cylindrical inner bore formed by a curved inner surface that is concentric with an outer surface of the cylinder. The flange is configured for restraining the barrel (such as with a practitioner's fingers) while the plunger is being forced into the barrel inner bore. The connector defines a passageway to place the barrel inner bore in fluid communication with another device, such as a cannula. The connector is configured to receive and form a fluid-tight seal with the other device.

The syringe plunger is configured with a shaft, an end flange at a proximal end of the shaft for pushing or pulling the plunger, and a sealing element at a distal end of the shaft. The shaft is comprised of a plurality of longitudinally extending shaft flanges connected at their lateral centers along their longitudinal lengths. The plunger is sized to extend through the length of the barrel inner bore, with it's sealing element adjoining the distal end of the barrel bore and it's proximal end extending out the proximal end of the barrel such that the plunger end flange may be pressed or pulled upon to insert or withdraw the plunger (with respect to the barrel inner bore) through the proximal end of the barrel. The sealing element is configured to form and maintain a hermetic seal within the inner surface of the barrel inner bore. Thus, as the plunger is withdrawn from the barrel bore, either fluid will be sucked into the inner bore via the connector, or a partial vacuum is formed within the inner bore between the connector and the sealing element.

Syringes typically come in different sizes, such as 3 cc, 10 cc, 12 cc, 20 cc, 35 cc and 60 cc. Some uses of a syringe (and particularly the larger syriunges), such as lyposuction, involve the drawing of tissue from the body of a subject using a cannula. In such a use, a practitioner can insert the plunger fully into the barrel inner bore, attach a proximal end of a cannula to the connector of the syringe, insert a distal end of the cannula (which has an opening) into the subject's body, draw the plunger out of the bore until the sealing portion is in the proximal end of the inner bore (to create a vacuum within the inner bore), and hold the plunger in that position with respect to the barrel while the distal end of the cannula is moved into contact with the desired tissue to be withdrawn from the subject. The holding of the plunger in place with respect to the barrel can be taxing on the practitioner, particularly while the practitioner is focused on the task of manipulating the distal end of the cannula within the patient.

Accordingly, there has existed a need for a device to hold a plunger in place with respect to a syringe barrel. Furthermore, there is a need for such a device to be both reliable and easy to use. Preferred embodiments of the present invention satisfy these and other needs, and provide further related advantages.

### SUMMARY

In various embodiments, the present invention solves some or all of the needs mentioned above, providing a reliable and easy to use clip configured to restrain the longitudinal movement of a plunger and thereby hold a sealing portion of a plunger in place with respect to a syringe barrel. In one form, the clip features a body defining a compression surface configured as part of a clamping mechanism to compress an outer surface of the barrel surrounding the sealing portion. This feature provides for the clip to compress the syringe barrel around a portion of the plunger, and thereby reliably lock the plunger in place with respect to the barrel.

In one variation, the clamping mechanism of the invention features a first compression arm forming the first compression surface, a second compression arm forming a second compression surface, and a connecting portion that connects proximal ends of the first and second compression arms, holding them in a substantially rigid position with respect to each other. These features provide for the clip to be applied to the barrel with relative simplicity.

In another variation, the clamping mechanism of the invention features a first compression arm forming the first compression surface, a second compression arm forming a second compression surface, and a connecting portion that connects proximal ends of the first and second compression arms, holding the proximal ends at a substantially constant distance from each other. This variation also features a latch hingedly attached to a distal end of the first compression arm, and having a locking-end configured to latch with a distal end of the second compression arm. These features provide a secure locking mechanism that will reliably remain in place during use.

In yet another variation, the body is a flexible strap, and the clamping mechanism features a latch configured to attached opposite ends of the strap such that the strap can latch to form a compression surface loop characterized by an inner circumference of less than a minimum distance around the outer surface of the syringe surrounding the sealing portion.

While the clip of the invention may be configured for use with standard syringe barrel configurations, other variations of the invention may include syringe barrels that are customized for use with the clips. For example, some variations of the invention may include as a feature that the barrel defines one or more notches forming surface(s) configured to receive the compression surface(s) of the clip.

Other variations of the invention pertain to a clip configured with first and second compression surfaces to directly compress opposite lateral sides of a longitudinally inserted plunger, such as on a longitudinally extending shaft flange, thus forming a stop that prevents the plunger from being sucked into a syringe barrel, and thereby holding the plunger's sealing portion in place. The invention may feature a third and fourth compression surface configured as part of a second clamping mechanism to compress additional opposite sides of the plunger, such as on a second longitudinally extending shaft flange, wherein the body further defines a latching mechanism configured both to lock the first and second compression surfaces and the third and fourth compression surfaces onto the plunger. These pairs of compression surfaces may be configured to compress the plunger at 90 degree angles to each other.

Additionally, these other variations may include a catch configured to lock onto a syringe flange when the clamping mechanism is clamped on to opposite sides of the plunger. Advantageously, this feature provides for the plunger to be locked in place with respect to the barrel, preventing it from coming out of the barrel.

Other features and advantages of the invention will become apparent from the following detailed description of the preferred embodiments, taken with the accompanying drawings, which illustrate, by way of example, the principles of the invention. The detailed description of particular preferred embodiments, as set out below to enable one to build and use an embodiment of the invention, are not intended to limit the enumerated claims, but rather, they are intended to serve as particular examples of the claimed invention.

### BRIEF DESCRIPTION OF THE DRAWINGS

FIG. 1 is a left side cross-sectional view of a first embodiment of a syringe under the invention.

FIG. 2 is a front cross-sectional view of the syringe depicted in FIG. 1, along with a clip under the invention, with the clip in position to be applied to the syringe.

FIG. 3 is a front cross-sectional view of the syringe and clip depicted in FIG. 2, with the clip applied to the syringe FIG. 4 is a partial right side cross-sectional view of a variation of the syringe depicted in FIG. 1.

FIG. 5 is a front cross-sectional view of the syringe depicted in FIG. 4.

FIG. 6 is a left side cross-sectional view of a second embodiment of a syringe under the invention.

FIG. 7 is a front cross-sectional view of the syringe depicted in FIG. 6, along with a clip under the invention, with the clip in position to be applied to the syringe.

FIG. 8 is a front view of a clip under a third or fourth embodiment of the invention.

FIG. 9 is a front view of a clip under a fifth or sixth embodiment of the invention.

FIG. 10A is a front cross-sectional view of a clip under a seventh embodiment of the invention.

FIG. 10B is a right side view of a clip depicted in FIG. 10A.

FIG. 11 is a left side cross-sectional view of the clip depicted in FIG. 10A, as attached to a syringe under the invention.

### DETAILED DESCRIPTION OF THE PREFERRED EMBODIMENTS

The invention summarized above and defined by the enumerated claims may be better understood by referring to the following detailed description, which should be read with the accompanying drawings. This detailed description of particular preferred embodiments of the invention, set out below to enable one to build and use particular implementations of the invention, is not intended to limit the enumerated claims, but rather, it is intended to provide particular examples of them.

Typical embodiments of the present invention reside in a syringe having a barrel and a plunger, and a clip. The clip is configured to prevent the plunger from longitudinally moving in to and/or out of the barrel. Thus, the syringe may conveniently be used as a vacuum source that provides the necessary suction for a cannula to draw tissue from a subject's body.

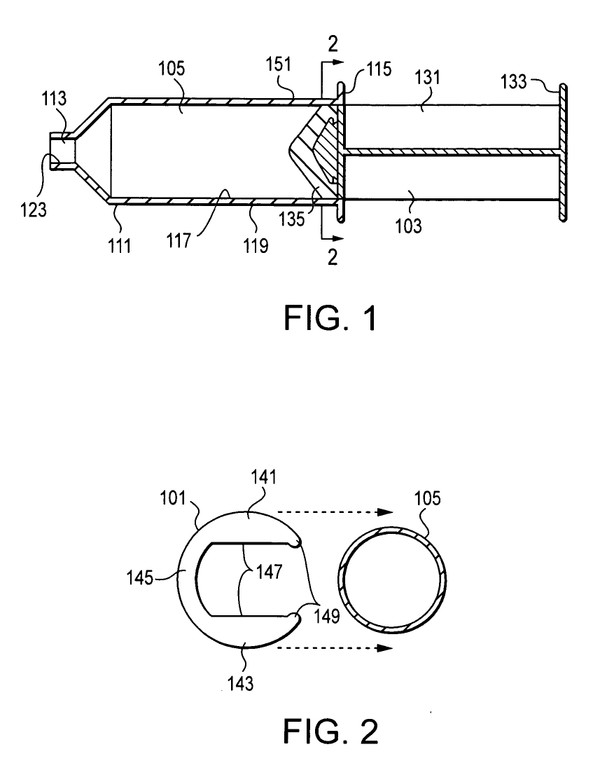

With reference to FIGS. 1 and 2, under a first embodiment of the present invention, a syringe may be provided with a clip 101 to lock a syringe plunger 103 in place within a syringe barrel cylinder 105. Using the clip to lock the plunger in place with respect to the barrel cylinder, the syringe may be used as a vacuum source, such as for use in lyposuction.

The syringe is configured with a barrel 111 forming the barrel cylinder 105, a connector 113 on a distal end of the barrel, and a barrel flange 115 on a proximal end of the barrel. The barrel cylinder is in the form of a cylinder defining an inner bore formed by an inner surface 117 that is concentric with an outer surface 119, and having the barrel flange at one end. The connector defines a passageway 123 placing the barrel inner bore in fluid communication with a cannula passageway, as is known in the art. The connector is configured to receive and form a fluid-tight seal with a cannula.

The syringe plunger 103 is configured with a shaft 131 comprising two or more longitudinally extending shaft flanges connected longitudinally along their lateral centers, an end flange 133 at a proximal end of the shaft, and a sealing element 135 at a distal end of the shaft. The plunger is sized to extend through the length of the barrel bore, with it's sealing element adjoining the distal end of the barrel bore and it's proximal end flange extending out the proximal end of the barrel bore such that the end flange may be pressed or pulled upon to insert or withdraw the plunger through the proximal end of the barrel. The sealing element is configured to form and maintain a hermetic seal within the barrel, even as the plunger is withdrawn from the barrel bore, potentially forming a vacuum within the barrel bore.

The clip 101 is a body that includes a first compression arm 141, a second compression arm 143, and a connecting portion 145. The first and second compression arms each define a compression surface 147. The first and second compression arm surfaces are preferably flat, parallel surfaces that face each other, and are spaced at a distance less than the diameter of the outer surface 119 of the barrel cylinder. Each compression arm further includes a lip 149 that protrudes from a distal end of its compression surface toward the opposite compression surface, thereby narrowing the distance between the compression surfaces.

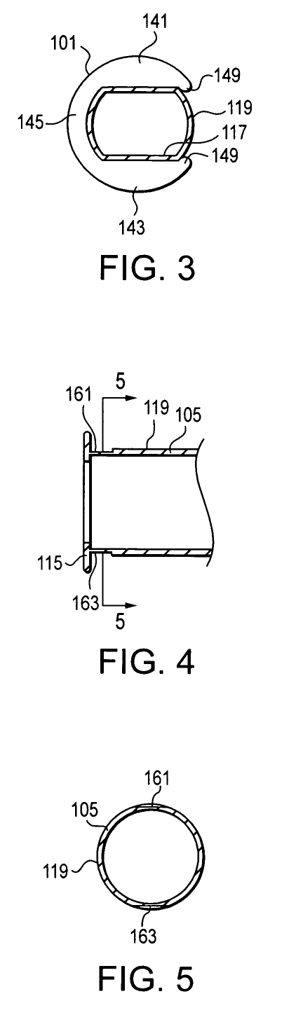

With reference to FIGS. 1 to 3, the connecting portion 145 connects proximal ends of the first and second compression arms 141 and 143, and holds them in a substantially rigid position with respect to each other, at a distance such that the compression surfaces 147 of the clip 101 will compress the barrel cylinder when the compression arms of the clip 101 are received over a clip reception portion 151 of the outer surface 119 of the barrel cylinder, as depicted in FIG. 3. The first and second compression arms thus form part of a clamping mechanism for the compression surfaces. The lips 149 narrow the opening at the distal ends of the compression arms, and are thereby configured to prevent an unforced removal of the clip from the barrel when the clip is received on the barrel.

As a result of the clip being received on the barrel, the barrel inner surface 117 may be slightly deformed from its undeformed cylindrical shape to a non-cylindrical shape. If, when the clip is received on the barrel, the sealing element 135 or the plunger shaft 131 is longitudinally positioned in the barrel such that it is within the reception portion 151 of the outer surface 119, then the deformed inner surface will compressibly hold the sealing element or plunger shaft, and thereby prevent the plunger 131 from moving longitudinally within the barrel cylinder 105. The reception portion may be a portion of the barrel where it is desirable to hold the sealing portion of the plunger. Preferably, the clip and the syringe are configured such that the sealing portion will maintain its seal when compressed within the deformed barrel inner surface.

With reference to FIGS. 4 and 5, in a variation of the first embodiment, the barrel cylinder 105 defines one or more notches on, and preferably a first notch 161 and a second notch 163 on opposite lateral sides of, the barrel cylinder outer surface 119 at a given longitudinal position. The first and second notches define a specific longitudinal location for the reception portion 151 of the barrel.

The first and second notches each form a flat surface, and these two flat surfaces are parallel to each other and normal to a line passing radially through a central axis of the barrel cylinder. More particularly, the notches form clip-reception surfaces on the clip reception portion of the barrel, and are configured to receive the first and second compression surfaces of the clip when the clip is received on the barrel. The reception portion and the first and second notches may be located at the proximal end of the barrel, adjacent to the barrel flange 115 (as depicted in FIG. 4), maximizing the quantity of tissue which may be drawn into the syringe due to the vacuum. Alternatively, the reception portion and the first and second notches may be located distally from the proximal end of the barrel, thereby avoiding the stiffness imparted to the barrel by the barrel flange.

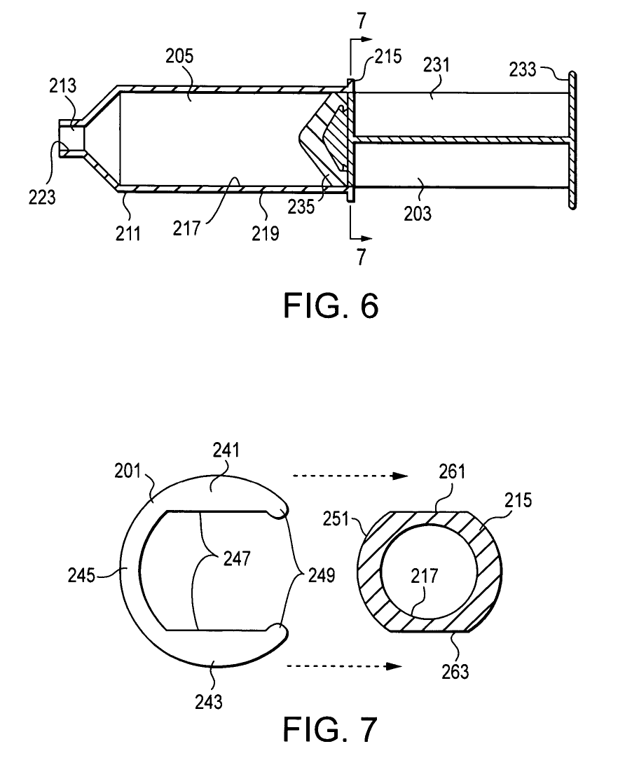

With reference to FIGS. 6 and 7, under a second embodiment of the present invention, a syringe may be provided with a clip 201 to lock a syringe plunger 203 in place within a syringe barrel cylinder 205. Using the clip to lock the plunger in place with respect to the barrel cylinder, the syringe may be used as a vacuum source, such as for use in lyposuction.

Similar to the first embodiment, the syringe is configured with a body 211 forming the barrel cylinder 205, a connector 213 at a distal end of the barrel, and a barrel flange 215 on a proximal end of the barrel. The barrel cylinder is in the form of a cylinder defining an inner bore formed by an inner surface 217 that is concentric with an outer surface 219, and having the barrel flange at one end. The connector defines a passageway 223 placing the barrel inner bore in fluid communication with a cannula passageway, as is known in the art. The connector is configured to receive and form a fluid-tight seal with a cannula.

The syringe plunger 203 is configured with a shaft 231 comprising two or more longitudinally extending shaft flanges connected at their lateral centers, an end flange 233 at a proximal end of the shaft, and a sealing element 235 at a distal end of the shaft. The plunger is sized to extend through the length of the barrel bore, with it's sealing element adjoining the distal end of the barrel bore and it's proximal end extending out the proximal end of the barrel such that the end flange may be pressed or pulled upon to insert or withdraw the plunger through the proximal end of the barrel. The sealing element is configured to form and maintain a hermetic seal within the barrel, even as the plunger is withdrawn from the barrel bore, potentially forming a vacuum.

The clip 201 is a body that includes a first compression arm 241, a second compression arm 243, and a connecting portion 245. The first and second compression arms each define a compression surface 247. The first and second compression arm surfaces are preferably flat, parallel surfaces that face each other, and are spaced at a distance less than the diameter of the outer surface 219 of the barrel cylinder. Each compression arm further includes a lip 249 that protrudes from a distal end of its compression surface toward the opposite compression surface, thereby narrowing the distance between the compression surfaces.

The connecting portion 245 connects proximal ends of the first and second compression arms 241 and 243, and holds them in a substantially rigid position with respect to each other, such that the compression surfaces 247 of the clip 201 will compress the barrel flange 215 when the compression arms of the clip 201 are received over the barrel flange, which defines a clip reception portion 251 of the barrel. The first and second compression surfaces of the compression arms thus form part of a clamping mechanism for the barrel. The lips 249 narrow the opening at the distal ends of the compression arms, and are thereby configured to prevent an unforced lateral removal of the clip from the barrel flange when the clip is received on the barrel flange.

The barrel flange 215 defines one or more notches on, and preferably a first notch 261 and a second notch 263 on opposite lateral sides of, the barrel flange 215. The first and second notches each form a circumferentially flat surface, and these two flat surfaces are parallel to each other and normal to a line passing radially through a central axis of the barrel cylinder. More particularly, the notches form clip-reception surfaces on the clip reception portion of the barrel, and are configured to receive the first and second compression surfaces of the clip when the clip is received on the barrel.

As a result of the clip being received on the barrel flange, the barrel inner surface 217 is deformed from its undeformed cylindrical shape to a non-cylindrical shape. If, when the clip is received on the barrel, the sealing element 235 or the plunger shaft 231 is longitudinally positioned in the barrel such that it is within the reception portion 251 of the barrel, then the deformed inner surface will compressibly hold the sealing element or plunger shaft, and thereby prevent the plunger 231 from moving longitudinally within the barrel. Preferably, the clip and the syringe are configured such that the sealing portion will maintain its seal when compressed within the deformed barrel inner surface.

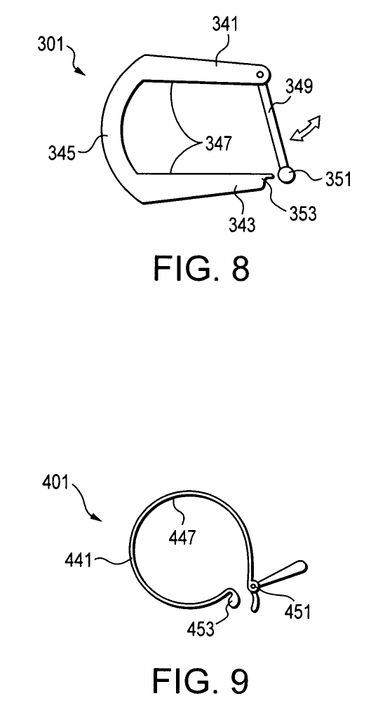

With reference to FIGS. 1 and 8, in a third and fourth embodiment of the invention, a clip 301 is formed from a body that includes a first compression arm 341, a second compression arm 343, and a connecting portion 345. The first and second compression arms each defined a compression surface 347. The first and second compression surfaces are preferably configured to be received on a barrel cylinder or barrel flange, as described with respect to the first and second embodiments, respectively. The connecting portion either is flexible in bending, providing for the compression arms to rotate away from one another approximately around their proximal ends, or is similarly hinged.

The connecting portion 345 connects proximal ends of the first and second compression arms 341 and 343, and is substantially stiff in extension, such that it holds them at an appropriate distance apart from each other for the clip to function. The first compression arm further includes a hingedly attached latch 349 having a locking-end 351 that is configured to mate (i.e., latch) with a locking flange 353 that protrudes from a distal end of the second compression arm. When the locking-end 351 is mated with the locking flange 353, the distal ends of the compression arms are held at the same distance apart as are the proximal ends, making the compression surfaces 347 parallel to one another.

The clip 301 is thereby configured such that it will compress the barrel if the compression surfaces 347 of the clip 301 are received around the reception portion 151 of the barrel, and the locking-end is mated with the locking flange 353. The first and second compression surfaces of the compression arms thus form part a clamping mechanism for the barrel. The latched latch 349 blocks the opening at the distal end of the locking arms, and thereby prevents removal of the clip from the barrel when the clip is so received on the barrel.

As described above for the first embodiment, in a variation of the third embodiment, the syringe barrel may define one or more notches on, and preferably a first notch and a second notch on opposite lateral sides of, the barrel outer surface at a given longitudinal position. The first and second notches define a specific longitudinal location for the reception portion of the barrel.

With reference to FIGS. 1 and 9, in a fifth and sixth embodiment of the invention, a clip 401 is a body that includes a flexible strap 441. Relative to the needed strength to function, the strap is high in tensile strength, but is flexible in bending. To that end, the strap may be composed of a variety of materials, such as metal, fabrics, and flexible composite materials. The strap defines a compression surface 447 to be received around the circumference of the barrel cylinder, or the barrel flange of the barrel described with respect to the first and second embodiments, respectively.

The clip further includes a latch having a locking-end 451 that is configured to mate (i.e., latch) with a locking flange 453 at an opposite end of the strap 441. When the locking-end 451 is mated with the locking flange 453, the strap forms a flexible loop having a smaller circumference than the outer surface circumference of the barrel at the reception portion.

The clip 401 is thereby configured such that it will radially compress the barrel if the compression surface 447 of the clip 401 is received around the reception portion 151 of the barrel, and the locking-end is mated with the locking flange 453. The compression surface thus forms part of a clamping mechanism for the barrel. The latched latch 449 prevents removal of the clip from the barrel when the clip is so received on the barrel.

In a first variation of the fifth and sixth embodiments, the clip may include a rigid arm forming a compression surface and a flexible strap that functions as part of a clamping mechanism to clamp the compression surface against the barrel. In both the originally described fifth and sixth embodiments and this variation, the clip includes a flexible strap, and the clamping mechanism further includes a latch configured to attached opposite ends of the clip such that the compression surface compresses the outer surface of the barrel around the sealing portion.

Similar to as described above for the first embodiment, in a second variation of the fifth embodiment, the syringe barrel may define one or more notches on the barrel cylinder outer surface at a given longitudinal position. The one or more notches define a specific longitudinal location for the reception portion of the barrel. Optionally, a single notch may extend around the entire barrel as a longitudinal region having a thinner barrel Wall thickness. Optionally, the latch may be configured such that the clip is always left in place in the notch (and/or on the barrel), and tightened down when the plunger is to be locked in place. This option may be used with a number of embodiments of the invention.

For each of the above-described embodiments, as a result of the clip being received on the barrel, the barrel inner surface 117 is deformed from its undeformed a cylindrical shape to a non-cylindrical shape, or in the case of the fourth embodiment, to a cylindrical shape of a reduced diameter. If, when the clip is received on the barrel, the sealing element 135 or plunger shaft is longitudinally positioned in the barrel such that it is within the reception portion 151, then the deformed inner surface will compressibly hold the sealing element or plunger shaft, and thereby prevent the plunger 131 from moving longitudinally within the barrel.

Prior to use, the plunger is fully inserted into the barrel bore. To create suction (i.e., i.e., a vacuum within the barrel), such as for a cannula, a user draws the plunger out of the bore, preferably to the point where the sealing portion is within the reception portion of the barrel. The clip is then placed on the reception portion, deforming the bore inner surface around the sealing portion of the plunger. The deformed inner surface clamps down on the sealing portion, locking the plunger in place.

As noted above, in the first and second embodiments of the invention, the clip is in the form of a substantially rigid c-shaped device. To place the clip on the reception portion of the barrel, the open end of the c-shaped clip is placed against one side of the reception portion, and radially pressed into place over the reception portion. The lips form fingers to prevent the clip from slipping longitudinally off the barrel without the direct application of force by the user.

In the third and fourth embodiments of the invention, the clip is in the form of a flexible c-shaped device having a lock that clamps the two compression arms together. To place the clip on the reception portion of the barrel, the open end of the c-shaped clip is placed over the reception portion. The compression arms of the clip are then squeezed together and the latch is latched to maintain the clips squeeze on the barrel. Optionally, the latch mechanism may include a mechanical lever usable to draw the two distal ends of the compression arms together while latching the latch. The latch prevents the clip from slipping longitudinally off the barrel.

In the fifth and sixth embodiments of the invention, the clip is in the form of a flexible device having a lock that clamps the ends of the device together. To place the clip on the reception portion of the barrel, the clip is flexibly bent open and then placed around the barrel. The latch mechanism includes a mechanical lever usable to draw the two ends together while latching the latch. The latch prevents the clip from slipping longitudinally off the barrel.

Other variations are also envisioned within the scope of the invention. For example, In the second, fourth and sixth embodiments of the invention, the barrel flange had flat notches. Nevertheless, notches of other configuration, and clips that are received on barrel flanges that lack notches, are also envisioned.

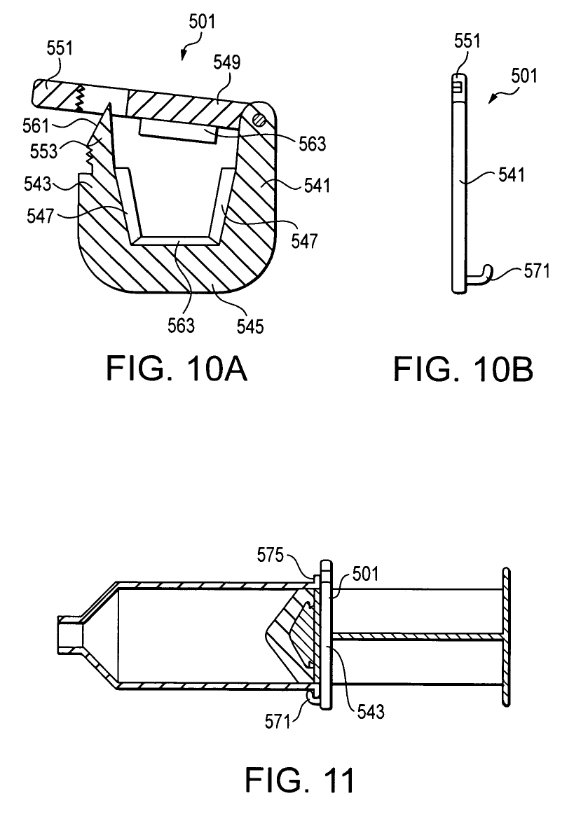

With reference to FIGS. 10A, 10B (front and right side views) and 11 (left side view with syringe in cross-section), a seventh embodiment of the invention is configured to clamp on to the plunger itself, rather than to the barrel. Similar to the third and fourth embodiments of the invention, a clip 501 is formed from a body that includes a first compression arm 541, a second compression arm 543, and a connecting portion 545. The first and second compression arms each defined a compression surface 547. However, in the seventh embodiment, the first and second compression surfaces are preferably configured as knife edges to be received on (and grippingly compress) opposite sides of a shaft flange. The typical plunger will include at least two shaft flanges extending across a width slightly less than the internal diameter of the barrel, preferably where two flanges are at ninety degree angles to each other, and where these two flanges intersect at their lateral center (along an axial centerline of the plunger).

The connecting portion 545 is preferably flexible in bending, providing for the compression arms to rotate away from one another approximately around their proximal ends, but may be similarly hinged. The connecting portion connects proximal ends of the first and second compression arms 541 and 543, and is substantially stiff in extension, such that it holds them at an appropriate distance apart from each other for the clip to function (i.e., lock on to a shaft flange of a plunger for which the clip is to be used). The first compression arm further includes a hingedly attached latch 549 having a locking-end 551 that is configured to mate (i.e., latch) with a locking flange 553 that protrudes from a distal end of the second compression arm.

The compression arms are configured such that when the latch is not latched, the compression arms extend slightly apart, making their distal ends farther apart than their proximal ends, and making their compression surfaces farther apart than the width of the shaft flange for which the clip is to be used. The latch mechanism, and preferably the locking flange, includes a ramp 561 configured to squeeze the distal ends of the compression arms together during a latching operation. Thus, when the locking-end 551 is mated with the locking flange 553, the distal ends of the compression arms are held at the same distance apart as are the proximal ends, making the compression surfaces 547 parallel to one another at a distance slightly less than the width of a shaft flange (so as to lock on to the shaft flange of the plunger for which the clip is to be used). The first and second compression surfaces of the compression arms thus form part a first clamping mechanism for the plunger.

The seventh embodiment is further configured with a second clamping mechanism that also clamps on to the plunger itself, rather than to the barrel. More particularly, the connecting portion 545 and the latch 549 each defined a second-clamping-mechanism compression surface 563 similar to the first-clamping-mechanism compression surfaces, these compression surfaces are preferably configured as knife edges to be received on (and grippingly compress) opposite sides of the second shaft flange (i.e., the shaft flange not grasped by the first clamping mechanism).

The first and second compression arms are substantially stiff in extension, such that they hold the connecting portion and the latch at an appropriate distance apart from each other for the clip to function (i.e., lock on to the second shaft flange of a plunger for which the clip is to be used). The latch mechanism is configured such that, when the latch is latched, the second-clamping-mechanism compression surfaces are parallel, and at a distance appropriate to lock onto the second shaft flange. Thus, when the latch is open the clip may be placed freely over both shaft flanges of the plunger, and when the latch is latched the clamping surfaces of the first and second clamping mechanisms moved together to clamp on to the first and second shaft flanges, respectively. When latched, the latch 549 both blocks the opening at the distal end of the locking arms, thereby preventing removal of the clip from the barrel when the clip is so received on the barrel, and contributes to the locking of the clip on the plunger.

With the clip locked on to the shaft flanges, the plunger will be locked from moving into the barrel past the point at which the clip adjoins the barrel flange. The clip further in includes features to lock the clip to the barrel flange, such that when the clip is locked on to the shaft flange, the plunger will also be locked for moving out of the barrel.

More particularly, the clip 501 further includes at least one protrusion in the form of a catch 571 adapted to receive and extend longitudinally around at least one edge of the barrel flange 575. In use, a practitioner will typically start with the plunger inserted deeply within a barrel. The practitioner will withdraw the plunger to a point in the barrel causing adequate vacuum for a given medical procedure. While holding the plunger in this partially withdrawn position with one hand, the practitioner will place the clip around the shaft flanges and adjoining the barrel flange such that the catch is received on and extends around the barrel flange, and the compression surfaces are loosely positioned around the shaft flanges. The practitioner then compresses the latch to a closed position, thereby locking the compression surfaces to the shaft flanges, and thereby locking the catch on to the barrel flange.

It is to be understood that the invention comprises related apparatus and methods for producing a vacuum within a syringe, as well as the apparatus and methods of use for the syringe itself. The above disclosed features can be combined in a wide variety of configurations within the anticipated scope of the invention.

While particular forms of the invention have been illustrated and described, it will be apparent that various modifications can be made without departing from the spirit and scope of the invention. Thus, although the invention has been described in detail with reference only to the preferred embodiments, those having ordinary skill in the art will appreciate that various modifications can be made without departing from the scope of the invention. Accordingly, the invention is not intended to be limited by the above discussion, and is defined with reference to the following claims.

What is claimed is:

1. A clip for restraining longitudinal movement of a plunger within a barrel of a syringe, the plunger having a sealing portion, comprising:
a body defining a compression surface configured as part of a clamping mechanism to compress an outer surface of the barrel surrounding the sealing portion.

2. The clip of claim 1, wherein the clamping mechanism comprises:
a first compression arm forming the first compression surface;
a second compression arm forming a second compression surface; and
a connecting portion that connects proximal ends of the first and second compression arms, holding them in a substantially rigid position with respect to each other.

3. The clip of claim 1, wherein the clamping mechanism comprises:
a first compression arm forming the first compression surface;
a second compression arm forming a second compression surface; and
a connecting portion that connects proximal ends of the first and second compression arms, holding the proximal ends at a substantially constant distance from each other; and
a latch hingedly attached to a distal end of the first compression arm, and having a locking-end configured to latch with a distal end of the second compression arm.

4. The clip of claim 1, wherein:
the body includes a flexible strap; and
the clamping mechanism further comprises a latch configured to attached opposite ends of the body such that the strap can latch to form a compression surface loop characterized by an inner circumference of less than a minimum distance around the outer surface of the syringe surrounding the sealing portion.

5. The clip of claim 1, wherein:
the body includes a flexible strap; and
the clamping mechanism further comprises a latch configured to attached opposite ends of the body such that the compression surface compresses the outer surface of the barrel around the sealing portion.

6. A vacuum system, comprising:
a syringe including a barrel and a plunger, the plunger having a sealing portion that is longitudinally movable within the barrel; and
the clip of claim 1.

7. The system of claim 6, wherein:
the barrel defines a notch forming a surface configured to receive the compression surface.

8. The system of claim 7, wherein the clip comprises:
a first compression arm forming the first compression surface;
a second compression arm forming a second compression surface; and
a connecting portion that connects proximal ends of the first and second compression arms, holding them in a substantially rigid position with respect to each other.

9. The system of claim 8, wherein:
the barrel defines a second notch forming a surface configured to receive the second compression surface.

10. A clip for restraining longitudinal movement of a plunger within a barrel of a syringe, the plunger having a first longitudinally extending flange and a second longitudinally extending flange, comprising:
a body defining a first compression surface and a second compression surface configured as part of a first clamping mechanism to compress opposite sides of the first plunger flange;
wherein the body further defines a third compression surface and a fourth compression surface configured as part of a second clamping mechanism to compress opposite sides of the second plunger flange; and
wherein the body further defines a latching mechanism configured both to lock the first and second compression surfaces onto the first plunger flange, and to lock the third and fourth compression surfaces onto the second plunger flange.

11. The clip of claim 10, wherein:
the syringe has a flange; and
the body further defines a catch configured to lock onto the syringe flange when the first clamping mechanism is clamped on to opposite sides of the plunger flange.

12. A vacuum system, comprising:
a syringe including a barrel and a plunger, the plunger having a first longitudinally extending flange and a second longitudinally extending flange; and
the clip of claim 11.

13. The clip of claim 1, wherein the clamping mechanism comprises a latch configured such that the clip may be left in place on the barrel, and tightened down when the plunger is to be restrained.

14. A clip for restraining longitudinal movement of a plunger within a barrel of a syringe, the plunger having a longitudinally extending flange, and the barrel having a flange, comprising:
a body defining a first compression surface and a second compression surface configured as part of a first clamping mechanism to compress and clamp on to opposite sides of the plunger flange;
wherein the body further defines a catch configured to lock onto the syringe flange when the first clamping mechanism is clamped on to opposite sides of the plunger flange.

15. A vacuum system, comprising:
a syringe including a barrel and a plunger, the plunger having a longitudinally extending flange, and the barrel having a flange; and
the clip of claim 14.

16. A method for restraining longitudinal movement of a plunger within a barrel of a syringe, the plunger having a sealing portion, comprising:
clamping a body defining a compression surface configured as part of a clamping mechanism, to compress an outer surface of the syringe surrounding the sealing portion.

17. A method for restraining longitudinal movement of a plunger within a barrel of a syringe, the plunger having a first longitudinally extending flange and a second longitudinally extending flange, comprising:
placing the clip of claim 10 such that the first compression surface and the second compression surface are positioned to compress opposite sides of the first plunger flange, and so that the third compression surface and the fourth compression surface are positioned to compress opposite sides of the second plunger flange; and
latching the latching mechanism.

18. A method for restraining longitudinal movement of a plunger within a barrel of a syringe, the plunger having a longitudinally extending flange, and the barrel having a flange, comprising:
clamping the clip of claim 14 onto the plunger such that the first compression surface and the second compression surface are positioned to compress opposite sides of the plunger flange, and so that the catch is locked onto the syringe flange.
